(19) United States
(12) Patent Application Publication
(10) Pub. No.: US 2022/0401457 A1
(43) Pub. Date: Dec. 22, 2022

(54) Use of Deoxycholic Acid, Derivatives, or Salts Thereof in Managing Bacterial Infections and Compositions Related Thereto

(71) Applicant: Emory University, Atlanta, GA (US)

(72) Inventor: Jorge Vidal, Lilburn, GA (US)

(21) Appl. No.: 17/639,318

(22) PCT Filed: Aug. 28, 2020
(86) PCT No.: PCT/US20/48519

§ 371 Date: Feb. 28, 2022

Related U.S. Application Data
(60) Provisional application No. 62/894,577, filed on Aug. 30, 2019.

(51) Int. Cl. A61K 31/565; A61K 9/00; A61K 31/573; A61K 47/10

(52) U.S. Cl. CPC A61K 31/565; A61K 9/0043; A61K 9/0053; A61K 31/573; A61K 47/10

(57) ABSTRACT

This disclosure relates to uses of deoxycholic acid, salts, or derivatives thereof in managing bacterial infections and compositions related thereto. In certain embodiments, this disclosure relates to methods of treating or preventing a bacterial infection comprising administering an effective amount of a deoxycholic acid, salts, or derivatives thereof to a subject in need thereof. In certain embodiments, this disclosure relates to methods of treating or preventing Streptococcus pneumoniae comprising administering an effective amount of a deoxycholic acid salt to a subject in need thereof.

### CROSS-REFERENCE TO RELATED APPLICATIONS

This application claims the benefit of U.S. Provisional Application No. 62/894,577 filed Aug. 30, 2019. The entirety of this application is hereby incorporated by reference for all purposes.

### BACKGROUND

Streptococcus pneumoniae (Spn) kills millions every year worldwide particularly those from the developing world. It colonizes the upper airways of most children. From the upper airways, Spn migrates to the ear epithelium causing otitis media. Antibiotic resistance in Spn strains is a serious concern. Therefore, there is a need for the development of therapeutic alternatives.

Sodium deoxycholate is a surfactant found in bile that participates in lipid digestion. Murray report a bile solubility test for identification of Streptococcus pneumoniae. J Clin Microbiol, 1979, 290-291. Le et al. report synergy between sodium deoxycholate and furazolidone against Enterobacteria. Cold Springs Harbor Laboratory, bioRXiv, 2019.

US Published Application No. 20140148429 to Hodge et al. report formulations of deoxycholic acid and salts thereof.

References cited herein are not an admission of prior art.

### SUMMARY

This disclosure relates to uses of deoxycholic acid, salts, or derivatives thereof in managing bacterial infections and compositions related thereto. In certain embodiments, this disclosure relates to methods of treating or preventing a bacterial infection comprising administering an effective amount of a deoxycholic acid, salts, or derivatives thereof to a subject in need thereof. In certain embodiments, this disclosure relates to methods of treating or preventing Streptococcus pneumoniae comprising administering an effective amount of a deoxycholic acid salt to a subject in need thereof.

In certain embodiments, an effective amount is a liquid, paste, or gel composition comprising deoxycholic acid, salts, or derivatives thereof at a concentration between 10.0 mg/mL to 0.1 mg/mL. In certain embodiments, an effective amount is between 1.0 and 0.3 mg/mL. In certain embodiments, an effective amount is between 0.6 and 0.4 mg/mL. In certain embodiments, an effective amount effective amount is about 0.5 mg/mL.

In certain embodiments, the deoxycholic acid, salt, or derivative thereof is administering in combination with a second antibiotic agent. In certain embodiments, the second antibiotic agent is selected from a penicillin, amoxicillin, azithromycin, amoxicillin and azithromycin, ampicillin, levofloxacin, macrolide, quinolone, doxycycline, tetracycline, clavulanic acid, clindamycin, cephalosporin, rifampin, vancomycin, fluoroquinolone, ceftriaxone, cefotaxime, ceftaroline, imipenem, linezolid, tigecycline, carbapenem, erythromycin, chloramphenicol, meropenem, sulfamethoxazole, trimethoprim, sulfamethoxazole and trimethoprim, or combinations thereof.

In certain embodiments, the subject is diagnosed as resistant to a second antibiotic such as erythromycin, chloramphenicol, meropenem, or tetracycline. In certain embodiments, the subject is diagnosed with bacterial pneumonia, meningitis, acute otitis media, otitis media with effusion, a bloodstream infection, ear infection or sinus infection.

In certain embodiments, the deoxycholic acid, salts, or derivatives thereof is administered by aerosol or spray in the pulmonary airway or nasal passage. In certain embodiments, the deoxycholic acid salt is administered intranasally or orally. In certain embodiments, the deoxycholic acid salt is delivered using a jet nebulizers, ultrasonic nebulizer, or vibrating mesh nebulizer. In certain embodiments, the deoxycholic acid salt is delivered to the lung by a syringe, nebulizer, or metered-dose inhaler. In certain embodiments, the deoxycholic acid salt is administered by absorption in the oral cavity. In certain embodiments, the deoxycholic acid salt is administered through the ear canal or tympanic membrane of the inner ear.

In certain embodiments, this disclosure contemplates pharmaceutical compositions comprising a deoxycholic acid, salt, or derivative thereof and optionally a second antibiotic and optionally a glucocorticoid. In certain embodiments, the deoxycholic acid, salt, or derivative thereof is at a concentration of less than 10.0 and greater than 0.1 mg/mL. In certain embodiments, the deoxycholic acid, salt, or derivative thereof is at a concentration of less than 1.0 and greater than 0.3 mg/mL. In certain embodiments, the deoxycholic acid, salt, or derivative thereof is at a concentration of about 0.5 mg/mL. In certain embodiments, the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate.

In certain embodiments, this disclosure contemplates a toothpaste comprising a deoxycholic acid, salt, or derivative thereof. In certain embodiments, the deoxycholic acid, salt, or derivative thereof is at a concentration of less than 10.0 and greater than 0.1 mg/mL. In certain embodiments, the deoxycholic acid, salt, or derivative thereof is at a concentration of less than 1.0 and greater than 0.3 mg/mL. In certain embodiments, the deoxycholic acid, salt, or derivative thereof is at a concentration of about 0.5 mg/mL. In certain embodiments, the composition further comprises an additional dispersing and/or viscosity modulating agent.

### BRIEF DESCRIPTION OF THE DRAWINGS

FIG. 1 illustrates the structure of deoxycholic acid and the sodium salt.

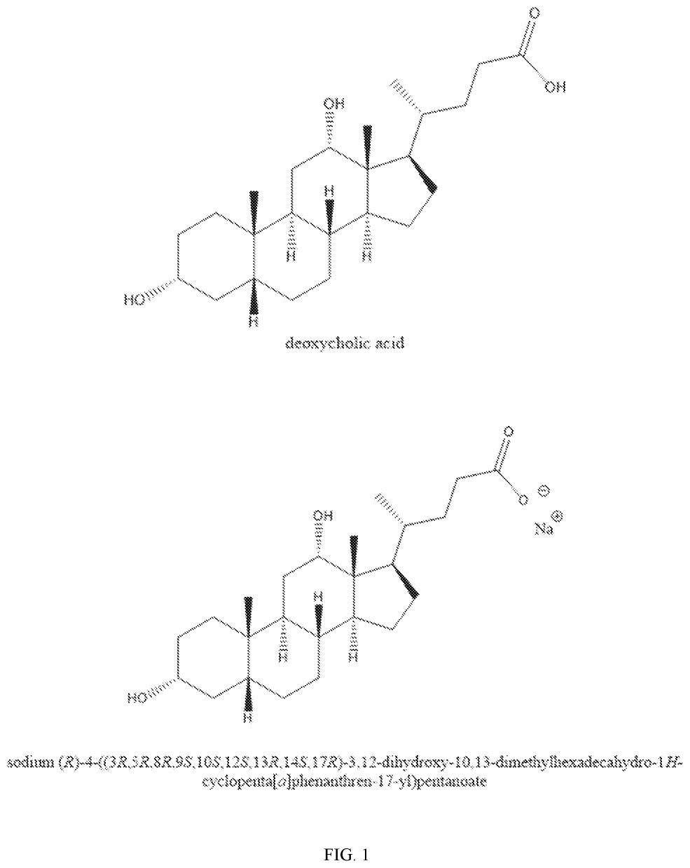

FIG. 2 shows data on the growth of Streptococcus pneumoniae strains, including several antibiotic resistant strains, when exposed to the sodium salt of deoxycholic acid at various concentrations.

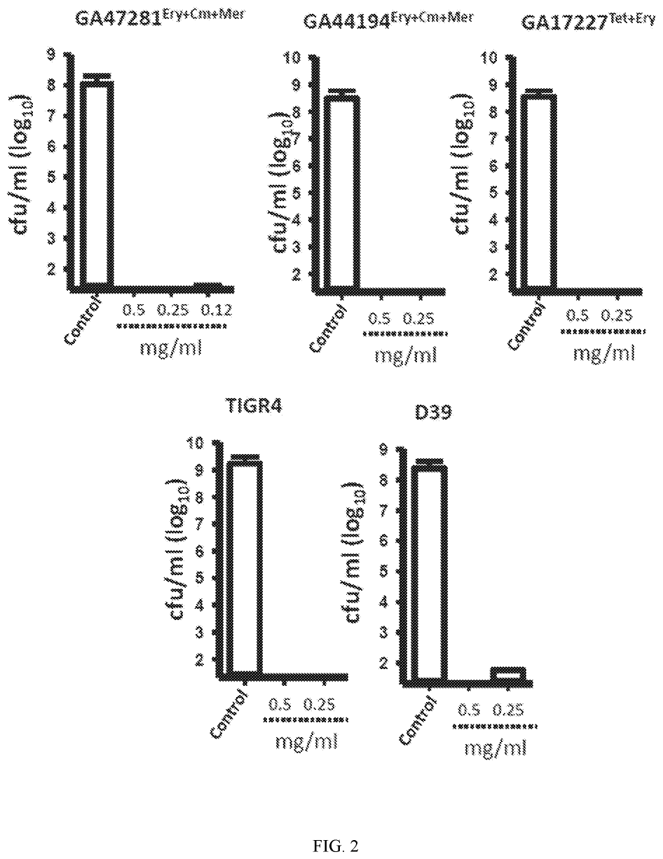

### DETAILED DESCRIPTION

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Embodiments of the present disclosure will employ, unless otherwise indicated, techniques of synthetic organic chemistry, biochemistry, biology, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used herein, “subject” refers any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In certain embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulphur atom or replacing an amino group with a hydroxyl group. The derivative may be a prodrug. Derivatives may be prepared by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

As used herein, the term “prodrug” refers a compound that, after administration, is metabolized (i.e., converted within the body) into a pharmacologically active drug. Examples include alkoxy esters of hydroxyl groups such as acetate esters, benzoate esters, alkyl ethers, amino acids esters, glycolic acid esters, malic acid esters, acyloxyalkyl esters, alkoxycarbonyloxy alkyl esters, S-acylthioalkyl esters, hydroxylamine amides, phosphonylmethoxy ethers, phosphates, phosphoramidates, and combinations thereof.

As used herein, the term “antibiotic” refers to a compound or group of compounds known to kill or slow the growth of bacteria. A variety of structural class are known. Examples include ampicillin, amikacin, ampicillin/sulbactam, amoxicillin/clavulanic acid, azithromycin, aztreonam, chloramphenicol, ceftriaxone, ceftazidime, clindamycin, cefotaxime, clarithromycin, cefazolin, ciprofloxacin, cefuroxime, erythromycin, ertapenem, nitrofurantoin, gentamicin, imipenem, levofloxacin, linezolid, meropenem, moxifloxacin oxacillin, penicillin, piperacillin, cefepime, piperacillin/tazobactam, rifampin, quinupristin/dalfopristin, tetracycline, tigecycline, tobramycin, trimethoprim/sulfamethoxazole, and vancomycin.

“Carrier materials” are excipients that are compatible with pharmaceutical formulations. Such carrier materials include, e.g., binders, suspending agents, disintegration agents, filling agents, surfactants, solubilizers, stabilizers, lubricants, wetting agents, diluents, and the like. Examples include, but are not limited to, acacia, gelatin, colloidal silicon dioxide, calcium glycerophosphate, calcium lactate, maltodextrin, glycerin, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

“Dispersing agents,” and/or “viscosity modulating agents” are materials that control the diffusion and homogeneity of an agent through liquid media. Examples of diffusion facilitators/dispersing agents include but are not limited to hydrophilic polymers, electrolytes, polyvinylpyrrolidone (PVP) and the carbohydrate-based dispersing agents such as, for example, hydroxypropyl celluloses, hydroxypropyl methylcelluloses, carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer, 4-(1,1,3,3-tetramethylbutyl)-phenolpolymer with ethylene oxide and formaldehyde (also known as tyloxapol), poloxamers; and poloxamines (block copolymer derived from sequential addition of propylene oxide and ethylene oxide to ethylenediamine), polyvinylpyrrolidone, polyvinylpyrrolidone/vinyl acetate copolymer polyethylene glycol, e.g., the polyethylene glycol has a molecular weight of about 300 to about 6000, or about 3350 to about 4000, or about 7000 to about 5400, sodium carboxymethylcellulose, methylcellulose, sodium alginate, gums, such as, e.g., gum tragacanth and gum acacia, guar gum, including xanthan gum, sugars, such as, sodium carboxymethylcellulose, methylcellulose, sodium carboxymethylcellulose, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also be used as dispersing agents. Dispersing agents useful in liposomal dispersions and self-emulsifying dispersions of the antimicrobial agents disclosed herein are dimyristoyl phosphatidyl choline, natural phosphatidyl choline from eggs, natural phosphatidyl glycerol from eggs, cholesterol and isopropyl myristate.

As used herein, “sterilization” refers to a process used to destroy or remove microorganisms that are present in a product or packaging. Any suitable method available for sterilization of objects and compositions is used. Available methods for the inactivation of microorganisms include, but are not limited to, the application of extreme heat, lethal chemicals, or gamma radiation or E-beam irradiation. In some embodiment, a process for the preparation of an formulation comprises subjecting the formulation to a sterilization method selected from heat sterilization, chemical sterilization, radiation sterilization or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

### Managing Bacterial Infections

This disclosure relates to uses of deoxycholic acid, salts, or derivatives thereof in managing bacterial infections and compositions related thereto. In certain embodiments, this disclosure relates to methods of treating or preventing a bacterial infection comprising administering an effective amount of a deoxycholic acid, salt, or derivative thereof to a subject in need thereof. In certain embodiments, this disclosure relates to methods of treating or preventing Streptococcus pneumoniae comprising administering an effective amount of a deoxycholic acid salt to a subject in need thereof.

In certain embodiments, an effective amount is a liquid, paste, or gel composition comprising deoxycholic acid, salts, or derivatives thereof at a concentration between 10.0 mg/mL to 0.1 mg/mL. In certain embodiments, an effective amount is between 1.0 and 0.3 mg/mL. In certain embodiments, an effective amount effective amount is 0.5 mg/mL.

In certain embodiments, the deoxycholic acid, salt, or derivative thereof is administering in combination with a second antibiotic agent. In certain embodiments, the second antibiotic agent is selected from a penicillin, amoxicillin, azithromycin, amoxicillin and azithromycin, ampicillin, levofloxacin, macrolide, quinolone, doxycycline, tetracycline, clavulanic acid, clindamycin, cephalosporin, rifampin, vancomycin, fluoroquinolone, ceftriaxone, cefotaxime, ceftaroline, imipenem, linezolid, tigecycline, carbapenem, erythromycin, chloramphenicol, meropenem, sulfamethoxazole, trimethoprim, sulfamethoxazole and trimethoprim, or combinations thereof.

In certain embodiments, the subject is diagnosed as resistant to a second antibiotic such as erythromycin, chloramphenicol, meropenem, or tetracycline. In certain embodiments, the subject is diagnosed with bacterial pneumonia, meningitis, acute otitis media, otitis media with effusion, a bloodstream infection, ear infection or sinus infection.

In certain embodiments, the deoxycholic acid, salt, or derivative thereof is administered by aerosol or spray in the pulmonary airway. In certain embodiments, the deoxycholic acid salt is administered intranasally or orally. In certain embodiments, the deoxycholic acid salt is delivered using a jet nebulizers, ultrasonic nebulizer, or vibrating mesh nebulizer. In certain embodiments, the deoxycholic acid salt is delivered to the lung by a syringe, nebulizer, or metered-dose inhaler. In certain embodiments, the deoxycholic acid salt is administered by absorption in the oral cavity. In certain embodiments, the deoxycholic acid salt is administered through the ear canal or placed by tympanic membrane of the inner ear.

In certain embodiments, this disclosure contemplates pharmaceutical compositions comprising a deoxycholic acid, salt, or derivative thereof and optionally a second antibiotic and optionally a glucocorticoid. In certain embodiments, the deoxycholic acid, salts, or derivatives thereof are at a concentration of less than 10.0 and greater than 0.1 mg/mL.

In certain embodiments, the deoxycholic acid, salt, or derivative thereof is at a concentration of less than 1.0 and greater than 0.3 mg/mL. In certain embodiments, the deoxycholic acid, salt, or derivative thereof is at a concentration of about 0.5 mg/mL. In certain embodiments, the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate.

Systemic administration for the treatment of disorders due to bacterial infection, e.g., may create a potential inequality in drug concentration with higher circulating levels in the serum, and lower levels in the target structures. As a result, fairly large amounts of drug are required to overcome this inequality in order to deliver sufficient, therapeutically effective quantities to the lungs, nose, throat, or ear. Further, bioavailability is often decreased due to metabolism of the drug by the liver. In addition, systemic drug administration may increase the likelihood of systemic toxicities and adverse side effects as a result of the high serum amounts required to effectuate sufficient local delivery to the target site. Systemic toxicities may also occur as a result of liver breakdown and processing of the therapeutic agents, forming toxic metabolites that effectively erase any benefit attained from the administered therapeutic.

To overcome the toxic and attendant undesired side effects of systemic delivery of deoxycholic acid, salts, or derivatives thereof and optionally other antibiotic agents (which may be toxic to cells), disclosed herein are methods and compositions for local delivery of deoxycholic acid, salts, or derivatives thereof and optionally other antibiotic agents to the lungs, nose, throat, or ear structures. In further or alternative embodiments, the controlled-release formulations are capable of being administered. In some embodiments, the controlled release formulation is applied via syringe and needle, wherein the needle is inserted and guided to the area of target site.

Because of the localized targeting of deoxycholic acid, salts, or derivatives thereof and optionally other antibiotic agents, the risk of adverse effects will be reduced as a result of treatment with previously characterized toxic or ineffective antibiotics. Localized administration of deoxycholic acid, salts, or derivatives thereof and optionally other antibiotic agents reduces the risk of development of resistance to antibiotics compared to the risk for development of antibiotic resistance when an antibiotic is administered systemically.

In certain embodiments, compositions described herein are effective for recurring otic diseases or conditions including, for example, recurring ear infections in children without the need for changing treatment regimens (e.g., in response to development of antibiotic resistance). Accordingly, also contemplated within the scope of the embodiments herein is the use of deoxycholic acid, salts, or derivatives thereof and optionally other antibiotic agents in the treatment of otic diseases or conditions including otitis externa, otitis media, Ramsay Hunt syndrome, otosyphilis, AIED, Meniere's disease, and vestibular neuronitis, including therapeutic agents that have been previously rejected by practitioners because of adverse effects or ineffectiveness of the antibiotic.

Also included within the embodiments disclosed herein is the use of additional media and/or agents in combination with the compositions containing deoxycholic acid, salts, or derivatives thereof and optionally other antibiotic agents disclosed herein. When used, such agents assist in the treatment of hearing or equilibrium loss or dysfunction resulting from an autoimmune disorder, including vertigo, tinnitus, hearing loss, balance disorders, infections, inflammatory response or combinations thereof. Accordingly, agents that ameliorate or reduce the effects of vertigo, tinnitus, hearing loss, balance disorders, infections, inflammatory response or combinations thereof are also contemplated to be used in combination with the formulations described herein.

In some embodiments, the composition comprises deoxycholic acid, salts, or derivatives thereof and optionally other antibiotic agents as an immediate release agent(s) wherein the immediate release salts of deoxycholate is or is not used as the controlled-release agent, but instead a different antimicrobial agent, an additional therapeutic agent, or a combination thereof. In some embodiments, the composition further comprises an additional therapeutic agent, including an additional antimicrobial agent, an anti-inflammatory agent, a corticosteroid, or combinations thereof. In another aspect, the additional therapeutic agent is an immediate release or a controlled release agent.

In some embodiments, provided herein are controlled release formulations of deoxycholic acid, salts, or derivatives thereof and optionally other antibiotic agents for local treatment, thereby avoiding side effects as a result of systemic administration. The locally administered deoxycholic acid, salts, or derivatives thereof and optionally other antibiotic agents are compatible with the lungs, nose, throat, ear structures, and are administered either directly to the desired structure, e.g. the tympanic cavity. By specifically targeting the structures, adverse side effects as a result of systemic treatment are avoided.

In some embodiments, the pharmaceutical formulations, compositions, or devices described herein are used in combination with (e.g., implantation, short-term use, long-term use, or removal of) implants (e.g., cochlear implants). As used herein, implants include cochlear implants, hearing sparing devices, hearing-improvement devices, short electrodes, tympanostomy tubes, micro-prostheses or piston-like prostheses; needles; stem cell transplants; drug delivery devices; any cell-based therapeutic; or the like. In some instances, the implants are used in conjunction with a patient experiencing hearing loss.

In some embodiments, a composition disclosed herein is administered to an individual in need thereof once. In some embodiments, a composition disclosed herein is administered to an individual in need thereof more than once. In some embodiments, a first administration of a composition disclosed herein is followed by a second administration of a composition disclosed herein. In some embodiments, a first administration of a composition disclosed herein is followed by a second and third administration of a composition disclosed herein. In some embodiments, a first administration of a composition disclosed herein is followed by a second, third, and fourth administration of a composition disclosed herein. In some embodiments, a first administration of a composition disclosed herein is followed by a second, third, fourth, and fifth administration of a composition disclosed herein.

The number of times a composition is administered to an individual in need thereof depends on the discretion of a medical professional, the disorder, the severity of the disorder, and the individual's response to the formulation. In some embodiments, a composition disclosed herein is administered once to an individual in need thereof with a mild acute condition. In some embodiments, a composition disclosed herein is administered more than once to an individual in need thereof with a moderate or severe acute condition. In the case wherein the patient's condition does not improve, upon the doctor's discretion the administration may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration may be given continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In one embodiment, the formulations disclosed herein additionally provides an immediate release of deoxycholic acid, salts, or derivatives thereof, or within 1 minute, or within 5 minutes, or within 10 minutes, or within 15 minutes, or within 30 minutes, or within 60 minutes or within 90 minutes. In other embodiments, a therapeutically effective amount of deoxycholic acid, salts, or derivatives thereof is released from the composition immediately, or within 1 minute, or within 5 minutes, or within 10 minutes, or within 15 minutes, or within 30 minutes, or within 60 minutes or within 90 minutes. In certain embodiments the composition comprises a pharmaceutically acceptable gel formulation providing immediate release of deoxycholic acid, salts, or derivatives thereof. Additional embodiments of the formulation may also include an agent that enhances the viscosity of the formulations included herein.

In certain embodiments, the formulation provides an extended release formulation deoxycholic acid, salts, or derivatives thereof. In certain embodiments, diffusion of deoxycholic acid, salts, or derivatives thereof the formulation occurs for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year. In other embodiments, a therapeutically effective amount of salts of deoxycholate is released from the formulation for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

In certain embodiments, the formulation provides a therapeutically effective amount of deoxycholic acid, salt, or derivative thereof at the site of disease with essentially no systemic exposure. In other embodiments, the formulation provides a therapeutically effective amount of deoxycholic acid, salt, or derivative thereof at the site of disease with little or no detectable systemic exposure.

### Formulations

In certain embodiments this disclosure relates to formulation of deoxycholic acid, salt, or derivative thereof for uses disclosed herein in the form of a liquid, paste, or gel composition at a concentration between 10.0 mg/mL to 0.1 mg/mL, 1.0 and 0.3 mg/mL or about 0.5 mg/mL. In certain embodiments this disclosure relates to formulation of deoxycholic acid, salt, or derivative thereof in the form of a liquid, paste, or gel composition at a concentration between 0.10% w/v to 0.01% w/v, 0.1% w/v and 0.03% w/v or about 0.05% w/v.

In certain embodiments, this disclosure relates to aqueous formulations of deoxycholic acid, salt, or derivative thereof at a concentration of from about 0.5 mg/mL to less than about 1.0 mg/mL and optionally a preservative, e.g., benzyl alcohol. In certain embodiments, the pH of the solution is about 8.1 to about 8.5, or about 7.5 to about 8.5.

In certain embodiments this disclosure relates to aqueous formulations of deoxycholic acid, salt, or derivative thereof at a concentration of from about 0.05% w/v to less than about 0.10% w/v and optionally a preservative effective amount of benzyl alcohol which formulations are stabilized against precipitation by adjusting the pH of the initially formed clear solution to a pH of from about 8.1 to about 8.5, or about 7.5 to about 8.5.

In certain embodiments, the aqueous solutions contain about 1% w/v of sodium chloride.

In certain embodiments, the pH is established by the use of a base. It is contemplated that any base can be used to increase the pH of the composition provided that it does not react with the deoxycholic acid, salt, or derivative thereof and will not cause harm to the patient. In some embodiments, the base is selected from the group consisting of metal carbonates, metal bicarbonates, metal hydroxides, or a mixture thereof. Examples of bases include, but are not limited to, a base selected from the group consisting of sodium carbonate, calcium carbonate, magnesium carbonate, zinc carbonate, sodium bicarbonate, sodium hydroxide and potassium hydroxide or a mixture thereof. In one embodiment, the base is sodium hydroxide.

In certain embodiments, the pH of the composition may be maintained at the desired pH during storage with the use of a buffer. Various buffers are known in the art and it is contemplated that any buffer having buffering capacity at the desired pH can be used in the formulations disclosed herein. In certain embodiments, the buffer is a phosphate buffer. The amount of phosphate in the composition can be determined to provide a desired pH and salt concentration. In certain embodiments, the composition comprises about 10 mM phosphate buffer. In certain embodiments, the composition comprises about 10 mM dibasic sodium phosphate buffer.

In certain embodiments, the composition comprises at least one excipient to aid in achieving a composition with desired properties, such as increased solubility, preservability or to provide an isotonic solution. In one embodiment, the composition comprises about 1% w/v sodium chloride. In another embodiment, the composition comprises about 0.9% w/v benzyl alcohol. In some embodiments, the composition comprises about 0.9% w/v benzyl alcohol and about 1% w/v sodium chloride.

In some embodiments, a composition disclosed herein is formulated to provide an ionic balance that is compatible with lung, throat, nose, or inner ear fluids (e.g., endolymph and/or perilymph). The osmolarity/osmolarity of a composition may be by measuring the osmolarity/osmolarity of the active agent and all excipients with the exception of any optional gelling and/or the thickening agent (e.g., polyoxyethylene-polyoxypropylene copolymers, carboxymethylcellulose or the like). In some instances, the practical osmolarity of a composition or device disclosed herein is measured by vapor pressure osmometry (e.g., vapor pressure depression method) that allows for determination of the osmolarity of a composition or device at higher temperatures. In some instances, vapor pressure depression method allows for determination of the osmolarity of a composition or device comprising a gelling agent at a higher temperature wherein the gelling agent is in the form of a gel.

In some embodiments, the osmolarity at a target site of action is about the same as the delivered osmolarity (i.e., osmolarity of materials that cross or penetrate to the target site) of a composition or device described herein. In some embodiments, a composition or device described herein has a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L.

In certain embodiments, a composition comprises deoxycholic acid, salt, or derivative thereof and a propellant. In certain embodiments, an aerosolizing propellant is compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane or combinations thereof. In certain embodiments, the disclosure contemplates a pressurized or unpressurized container comprising deoxycholic acid, salt, or derivative thereof. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, a deoxycholic acid, salt, or derivative thereof is in the form of an inhaled dosage. In this embodiment, the deoxycholic acid, salt, or derivative thereof may be in the form of an aerosol suspension, a dry powder or liquid particle form. The compounds may be prepared for delivery as a nasal spray or in an inhaler, such as a metered dose inhaler. In certain embodiments, the aerosol suspension is solution of deoxycholic acid, salt, or derivative thereof at a concentration of from about 0.5 mg/mL. In certain embodiments, the solution is aqueous. In certain embodiments, the solution is a halogenated propellant. Pressurized metered-dose inhalers (“MDI”) generally deliver aerosolized particles suspended in chlorofluorocarbon propellants such as CFC-11, CFC-12, or the non-chlorofluorocarbons or alternate propellants such as the fluorocarbons, HFC-134A or HFC-227 with or without surfactants and suitable bridging agents. Dry-powder inhalers can also be used, either breath activated or delivered by air or as pressure such as the dry-powder inhaler

In certain embodiments, composition disclosed herein, e.g., pharmaceutical composition, can further comprise a second therapeutic agent selected from the group consisting of: anti-microbial agents, vasoconstrictors, anti-thrombotic agents, anti-coagulation agents, anti-depressants, anti-inflammatory agents, analgesics, dispersion agents, anti-dispersion agents, penetration enhancers, steroids, tranquilizers, muscle relaxants, and anti-diarrhea agents. In some embodiments, a solution is in a container that contains up to 500 mL of solution. Such container can be a syringe or syringe-loadable container. In certain embodiments, the container is pliable thus allowing one to squeeze the solution out of the tip or opening of the container such that droplets readily form.

In certain embodiments, this disclosure relates to compositions comprising agents disclosed herein impregnated into gels and pastes disclosed herein optionally comprising biodegradable polymers. In certain embodiments, this disclosure relates to compositions comprising agents disclosed herein and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose is utilized as the gelling agent. In certain embodiments, the viscosity enhancing agents described herein are also utilized as the gelling agent for the gel formulations presented herein.

Useful gel formulations are considered to fall within the scope of the present disclosure. For example, other glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the formulations described herein. In some embodiments, the gels include, but are not limited to, alginate hydrogels, acemannan hydrogels and glycerin gels.

Polymers composed of polyoxypropylene and polyoxyethylene form thermoreversible gels when incorporated into aqueous solutions. These polymers have the ability to change from the liquid state to the gel state at temperatures close to body temperature, therefore allowing useful formulations that are applied to the targeted structure(s). The liquid state-to-gel state phase transition is dependent on the polymer concentration and the ingredients in the solution.

In certain embodiments, a biodegradable drug carrier may comprise ABA-type or BAB-type triblock copolymers or mixtures thereof, wherein the A-blocks are relatively hydrophobic and comprise biodegradable polyesters or poly(orthoester)s, and the B-blocks are relatively hydrophilic and comprise polyethylene glycol (PEG), said copolymers having a hydrophobic content. The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, epsilon-caprolactone, epsilon-hydroxyhexanoic acid, gamma-butyrolactone, gamma-hydroxybutyric acid, delta-valerolactone, delta-hydroxyvaleric acid, hydroxybutyric acids, malic acid, and copolymers thereof and having an average molecular weight of between about 600 and 3000 Daltons. The hydrophilic B-block segment is preferably polyethylene glycol (PEG) having an average molecular weight of between about 500 and 2200 Daltons. One contemplated constructs are poly(ethylene glycol-b-(DL-lactic acid-co-glycolic acid)-b-ethylene glycol), PEG-PLGA-PEG triblock copolymers.

Poloxamers are a nonionic surfactant composed of polyoxyethylene-polyoxypropylene copolymers having the general chemical formula,

H(OCH₂CH₂)ₐ(OCH(CH₃)CH₂)_{b}(OCH₂CH₂)ₐOH,

wherein a is an average number of oxy-ethylene monomers and b is an average number of oxy-propylene monomers. Poloxamer 407 (PF-127) has an average molar mass of 13,000. It contains approximately 70% ethylene oxide, which accounts for its hydrophilicity. Other poloxamers include 188 (F-68 grade), 237 (F-87 grade), 338 (F-108 grade). Concentrated solutions (>20% w/w) of the PF-127 copolymer are transformed from low viscosity transparent solutions to solid gels on heating to body temperature. This phenomenon, therefore, suggests that when placed in contact with the body, the gel preparation will form a semi-solid structure and a sustained release depot. Furthermore, PF-127 has good solubilizing capacity and low toxicity.

Since the polymer systems of thermoreversible gels dissolve more completely at reduced temperatures, methods of solubilization include adding the required amount of polymer to the amount of water to be used at reduced temperatures. Generally after wetting the polymer by shaking, the mixture is capped and placed in a cold chamber in order to dissolve the polymer. The mixture is stirred or shaken to bring about a more rapid dissolution of the thermoreversible gel polymer. The antimicrobial agent and various additives such as buffers, salts, and preservatives are subsequently added and dissolved. In some instances the antimicrobial agent and/or other pharmaceutically active agent is suspended if it is insoluble in water. The pH is modulated by the addition of appropriate buffering agents.

In certain embodiments, this disclosure relates to compositions comprising agents disclosed herein dispensed as a controlled-release foam. Examples of suitable foamable carriers for use in the compositions disclosed herein include, but are not limited to, alginate and derivatives thereof, carboxymethylcellulose and derivatives thereof, collagen, polysaccharides, including, for example, dextran, dextran derivatives, pectin, starch, modified starches such as starches having additional carboxyl and/or carboxamide groups and/or having hydrophilic side-chains, cellulose and derivatives thereof, agar and derivatives thereof, such as agar stabilized with polyacrylamide, polyethylene oxides, glycol gelatin, gums such as guar, karaya, tragacanth and locust bean gum, or combinations thereof. Also suitable are the salts of the aforementioned carriers, for example, sodium alginate. The formulation optionally further comprises a foaming agent, which promotes the formation of the foam, including a surfactant or external propellant. Examples of suitable foaming agents include cetrimide, lecithin, soaps, silicones and the like.

In certain embodiments, this disclosure relates to compositions comprising agents disclosed herein dispensed as a paint. As used herein, paints (also known as film formers) are solutions comprised of a solvent, a monomer or polymer, an active agent, and optionally one or more pharmaceutically-acceptable excipients. After application to a tissue, the solvent evaporates leaving behind a thin coating comprised of the monomers or polymers, and the active agent. The coating protects active agents and maintains them in an immobilized state at the site of application. This decreases the amount of active agent which may be lost and correspondingly increases the amount delivered to the subject. By way of non-limiting example, paints include collodions, and solutions comprising saccharide siloxane copolymers and a cross-linking agent. Collodions are ethyl ether/ethanol solutions containing pyroxylin (a nitrocellulose). After application, the ethyl ether/ethanol solution evaporates leaving behind a thin film of pyroxylin. In solutions comprising saccharide siloxane copolymers, the saccharide siloxane copolymers form the coating after evaporation of the solvent initiates the cross-linking of the saccharide siloxane copolymers. The paints contemplated for use herein, are flexible such that they do not interfere with the propagation of pressure waves through the ear. Further, the paints may be applied as a liquid (i.e. solution, suspension, or emulsion), a semisolid (i.e. a gel, foam, paste, or jelly) or an aerosol.

In certain embodiments, this disclosure contemplates a toothpaste comprising a deoxycholic acid, salt, or derivative thereof. In certain embodiments, this disclosure contemplates a toothbrush comprising a deoxycholic acid, salt, or derivative thereof. In certain embodiments, the toothpaste is between 20%-50% water by weight. In certain embodiments, the toothpaste further comprises a fluorine salt such as sodium fluoride, sodium monofluorophosphate or stannous fluoride. In certain embodiments, the toothpaste further comprises glycerol, sorbitol, xylitol, 1,2-propylene glycol, polyethene glycol, or combinations thereof. In certain embodiments, the toothpaste further comprises aluminum hydroxide, calcium carbonate, a calcium hydrogen phosphate, a silica, a zeolite, hydroxyapatite, or combinations thereof. In certain embodiments, the toothpaste further comprises sodium lauryl sulfate. In certain embodiments, the toothpaste further comprises triclosan, zinc chloride, or a combination thereof. In certain embodiments, the toothpaste further comprises sodium tripolyphosphate.

The disclosure also provides kits for preventing, treating or ameliorating the symptoms of a disease or disorder in a mammal. Such kits generally will comprise one or more compositions comprising deoxycholic acid, salt, or derivative thereof or devices disclosed herein, and instructions for using the kit. The disclosure also contemplates the use of one or more of compositions of deoxycholic acid, salt, or derivative thereof, in the manufacture of medicaments for treating, abating, reducing, or ameliorating the symptoms of a disease, dysfunction, or disorder in a mammal, such as a human that has, is suspected of having, or at risk for developing an lung, nose, throat, or ear disorder due to a bacterial infection.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of deoxycholic acid salt formulations compositions provided herein are contemplated as are a variety of treatments for any disease, disorder, or condition that would benefit by controlled release administration of deoxycholic acid, salts, or derivatives thereof to the lung, nose, or ear.

In some embodiments, a kit includes one or more additional containers, each with one or more of various materials (such as reagents, optionally in concentrated form, and/or devices) desirable from a commercial and user standpoint for use of a formulation described herein. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, an aseptic container is sealed with a cap comprising deoxycholic acid, salts, or derivatives thereof. In certain embodiments, the cap further comprises a septum secured against the aseptic container by the aluminum frame.

### EXAMPLES

Streptococcus pneumoniae (Spn) colonizes the upper airways of most children. From the upper airways, Spn migrates to the ear epithelium causing otitis media, a main cause of child disease in the US. Resistance in Spn strains is a serious concern. Bile salts produced in the human liver and secreted into the gut, including sodium deoxycholate. Sodium deoxycholate at a concentration of 10 mg/mL is an injectable treatment to reduce fat under the chin. The antibacterial potency of sodium deoxycholate against Spn was assessed. Experiments indicate that at a concentration as low as 0.5 mg/ml, sodium deoxycholate completely killed cultures of numerous different Spn strains within two hours of incubation, including vaccine strains and Spn strains with multiple resistance to antibiotics. Most tested pneumococcal strains were reference strains provided by the Centers for Disease Control and Prevention (CDC). Experiments mimicking pneumococcal colonization of the human airways showed that 0.5 mg/ml of sodium deoxycholate eradicated pneumococcal colonization that was otherwise at a very high density of 1×10⁷ cfu/mL pneumococci within only 10 minutes of exposure. It is contemplated that sodium deoxycholate can be used as a prophylactic agent to eradicate Spn from the upper airways and/or as a therapeutic agent to treat pneumococcal otitis media. To eradicate Spn from the upper airways of healthy children, a strategy using the following routes can be pursued (1) the use of nasal spray to impregnate the upper airways with sodium deoxycholate, or (2) as an additive to a gel or toothpaste. For its therapeutic used in the treatment of otitis media, ototopical antibiotic preparations can be added with sodium deoxycholate to kill antibiotic resistance Spn strains.

### Sodium Deoxycholate

One adds deoxycholic acid (DCA) to a basic solution of anhydrous dibasic sodium phosphate and NaOH. The solution is adjusted to a pH 8.3. One adds appropriate amounts of water and NaCl to increase osmolality up to 305 mOsm. One optionally lyophilizes the solution providing a product for reconstitution by addition of the appropriate amount of sterile water.

### Sodium Deoxycholate Gel

One mixes micronized deoxycholic acid or sodium deoxycholate, sodium phosphate dibasic, sodium phosphate monobasic monohydrate, sodium chloride, and sterile filtered DI water. One adjusts the pH to 8.3. One chills the buffer solution. One adds poloxamer 407 into the chilled solution with mixing. One filters the poloxamer solution using a sterile filter.

### Sodium Deoxycholate Gel Comprising Dexamethasone

One mixes micronized deoxycholic acid or sodium deoxycholate, micronized dexamethasone sodium phosphate dibasic, sodium phosphate monobasic monohydrate, sodium chloride, and sterile filtered deionized water. One adjusts the pH to 8.3. One chills the buffer solution. One adds poloxamer 407 into the chilled solution with mixing. One filters the poloxamer solution using a sterile filter.

1. A method of treating or preventing Streptococcus pneumoniae comprising administering an effective amount of a deoxycholic acid salt to a subject in need thereof.

2. The method of claim 1, wherein the effective amount is a liquid, paste, or gel composition at a concentration between 10.0 mg/mL to 0.1 mg/mL.

3. The method of claim 1, wherein the effective amount is between 1.0 and 0.3 mg/mL.

4. The method of claim 1, wherein the effective amount is 0.5 mg/mL.

5. The method of claim 1, wherein the deoxycholic acid salt is administering in combination with a second antibiotic agent.

6. The method of claim 1, wherein the second antibiotic agent is selected from a penicillin, amoxicillin, azithromycin, amoxicillin and azithromycin, ampicillin, levofloxacin, macrolide, quinolone, doxycycline, tetracycline, clavulanic acid, clindamycin, cephalosporin, rifampin, vancomycin, fluoroquinolone, ceftriaxone, cefotaxime, ceftaroline, imipenem, linezolid, tigecycline, carbapenem, erythromycin, chloramphenicol, meropenem, sulfamethoxazole, trimethoprim, sulfamethoxazole and trimethoprim, or combinations thereof.

7. The method of claim 1, wherein the subject is diagnosed as resistant to erythromycin, chloramphenicol, meropenem, or tetracycline.

8. The method of claim 1, wherein the subject is diagnosed with bacterial pneumonia, meningitis, acute otitis media, otitis media with effusion, a bloodstream infection, ear infection or sinus infection.

9. The method of claim 1, wherein the deoxycholic acid salt is administered by aerosolization or spray in the pulmonary airway.

10. The method of claim 1, wherein the deoxycholic acid salt is administered intranasally or orally.

11. The method of claim 1, wherein the deoxycholic acid salt is delivered using a jet nebulizers, ultrasonic nebulizer, or vibrating mesh nebulizer.

12. A composition comprising a deoxycholic acid salt is at a concentration of less than 10.0 and greater than 0.1 mg/mL.

13. The composition of claim 12, wherein a deoxycholic acid salt is at a concentration of less than 1.0 and greater than 0.3 mg/mL.

14. The composition of claim 12, wherein a deoxycholic acid salt is at a concentration of about 0.5 mg/mL.

15. The composition of claim 12, further comprising a second antibiotic agent.

16. The composition of claim 12, further comprising a fluorine salt.

17. The composition of claim 12, further comprising glycerol, sorbitol, xylitol, 1,2-propylene glycol, polyethene glycol, or combinations thereof.

18. The composition of claim 12, further comprising aluminum hydroxide, calcium carbonate, a calcium hydrogen phosphate, a silica, a zeolite, hydroxyapatite, or combinations thereof.

19. The composition of claim 12, further comprising a glucocorticoid.

20. The composition of claim 19, wherein the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate.
